(19) United States
(12) Patent Application Publication
(10) Pub. No.: US 2010/0057019 A1
(43) Pub. Date: Mar. 4, 2010

(54) DISTAL SECTION FOR MONORAIL CATHETER

(75) Inventor: Robert Zelenka, Milipitas, CA (US)

Correspondence Address: GRAYBEAL JACKSON LLP, 400 - 108TH AVENUE NE, SUITE 700, BELLEVUE, WA, 98004, US

(73) Assignee: Silicon Valley Medical Instruments, Inc., Fremont, CA (US)

(21) Appl. No.: 12/547,972

(22) Filed: Aug. 26, 2009

Related U.S. Application Data
(60) Provisional application No. 61/092,007, filed on Aug. 26, 2008.

(51) Int. Cl. A61M 25/00

(52) U.S. Cl. 604/264

(57) ABSTRACT

A catheter has a distal section comprising a distal tip section having a distal tip. The distal tip has a longitudinal axis. The distal section further includes a distal shaft having a longitudinal axis. The distal tip longitudinal axis is substantially parallel to and offset from the distal shaft longitudinal axis.

### PRIORITY CLAIM

The present application claims the benefit of copending U.S. Provisional Patent Application Ser. No. 61/092,007, filed Aug. 26, 2008, which application is incorporated herein by reference in its entirety.

### BACKGROUND OF THE INVENTION

The present invention relates generally to intravascular catheters. The present invention more specifically relates to improvements in distal section construction of monorail catheters The present invention still further relates to improvements in intravascular imaging catheters having monorail designs.

Atherosclerosis is a common disease that leads to lesion formation at multiple anatomical sites, including coronary and peripheral arteries. Progression of atherosclerotic lesions may lead to narrowing of the vascular lumen. Rupture of atherosclerotic lesions may lead to occlusion of blood flow. Restriction and occlusion of blood flow are serious patient risks.

The use of intravascular catheters for diagnosis and therapy of vessel disease is common. Catheters having short monorail (or short rapid exchange) designs are commonly used during such procedures. Monorail catheters are comprised of a distal section that includes a polymer distal shaft bonded to a polymer distal tip. The distal shaft has a lumen that may have internal working elements, such as an imaging core. The distal tip is typically softer than the distal shaft and receives guide wires through a short lumen. The guide wire exits the proximal port of the distal tip typically at an angle relative to the axis of the distal shaft. Long monorail catheter designs with a guide wire lumen parallel to the distal shaft lumen are described in U.S. Pat. No. 5,201,316 by Pomeranz et al., but have disadvantages that limit catheter performance compared to short monorail catheters.

Material is typically added between the distal shaft lumen and distal tip lumen to stiffen the joining section. Short monorail catheter designs are further described for example in U.S. Pat. No. 5,443,457 by Ginn et al. and U.S. patent application Ser. No. 11/963,596 by Zelenka et al. Such short monorail catheter designs have proven to be valuable for delivering catheters to small blood vessels (e.g., coronary arteries). However, certain design choices have limited catheter performance.

The angled exit of a guide wire from the distal tip increases friction between the guide wire and the catheter tip. Increased friction reduces the pushability of a catheter, or ability to transmit a force from the proximal end to the distal end of the catheter. Reduced pushability makes delivering a catheter to an anatomical site of interest more difficult. The angled exit of the guide wire from the distal tip further increases the profile of the catheter and guide wire. The larger profile reduces the trackability of a catheter, or ability to navigate a tortuous path.

A method to reduce friction between the guide wire and catheter tip is to line the distal tip lumen with a low-friction insert as described by example in U.S. Pat. No. 5,330,444 by Webler et al. Polytetrafluoroethylene (PTFE) is described as suitable for the low-friction insert. PTFE has long been used to line catheters because of its high lubricity as described by example in U.S. Pat. No. 4,636,346 by Gold et al.

The distal section remains susceptible to kinking (or prolapsing) depending on the type of material and length of the stiffened section. The added material also increases the distance between the distal shaft lumen, wherein an imaging core may be, and the distal tip. This limits how distal an imaging catheter is able to image.

In view of the limitations to catheter performance noted above, there is a need in the art for the design and construction of a catheter distal section that improves the pushability, trackability, and kink resistance of the catheter. It is also a need to be able to reduce the distance between the distal end of the imaging window of imaging catheters and the distal tip to enable imaging of more distal sites.

### SUMMARY OF THE INVENTION

In one embodiment, a catheter has a distal section comprising a distal tip section having a distal tip. The distal tip has a longitudinal axis. The distal section further includes a distal shaft having a longitudinal axis. The distal tip longitudinal axis is substantially parallel to and offset from the distal shaft longitudinal axis.

The distal shaft may be formed from at least one layer of material. The distal shaft may include a lumen for containing a working element. The working element may comprise a mechanically rotatable ultrasound imaging core. The working element may comprise a mechanically rotatable optical imaging core.

The distal tip section may include a guide wire lumen for receiving a guide wire. The longitudinal axis of the distal tip may also be the longitudinal axis of the guide wire lumen. The distal shaft may include a flushing vent hole arranged to be adjacent to the guide wire to enable the guide wire to prevent a flushing stream from the flushing vent hole from impinging a sidewall of a vessel in which the catheter is used. The distal tip section may have a length of 15 mm or less.

### BRIEF DESCRIPTION OF THE DRAWINGS

The features of the present invention which are believed to be novel are set forth with particularity in the appended claims. The invention, together with further features and advantages thereof, may best be understood by making reference to the following descriptions taken in conjunction with the accompanying drawings, in the several figures of which like reference numerals identify identical elements, and wherein:

FIG. 1 is a side view of a prior art catheter distal section, shown in cross section;

FIG. 2 is a side view, in section, of a prior art catheter distal section with guide wire;

FIG. 3 is a side view, in section, of a prior art catheter distal section with guide wire in a vessel;

FIG. 4 is a side view, in section, of the distal end of a catheter and guide wire according to one embodiment of the present invention;

FIG. 5 is a side view, in section, illustrating the catheter distal tip of the catheter of FIG. 4 in greater detail; and

FIG. 6 is a side view, in section, of the distal end of a catheter and guide wire according to another embodiment of the present invention.

### DETAILED DESCRIPTION OF THE INVENTION

A catheter is a common medical device and has a flexible tubular body having a proximal end and a distal end. The catheter comprises a proximal section and a distal section. The distal section is generally formed by extrusion of thermoplastics such as polyethylene, nylon, or a polyether block amide (Pebax®). The distal section of a catheter according to various embodiments of the present invention comprises a distal shaft having a working lumen and a distal tip having a guide wire lumen. The distal shaft may also be formed of more than one layer. The distal shaft has a working lumen that may extend from the proximal end of the catheter to the distal tip.

The distal tip has a short monorail design and is generally formed over the distal most 0.5 cm to 3 cm of the distal section. The distal tip is generally less stiff than the distal shaft. A distal entry port for the guide wire lumen is generally located at the distal tip. An exit port of the guide wire lumen is proximal to the distal entry port.

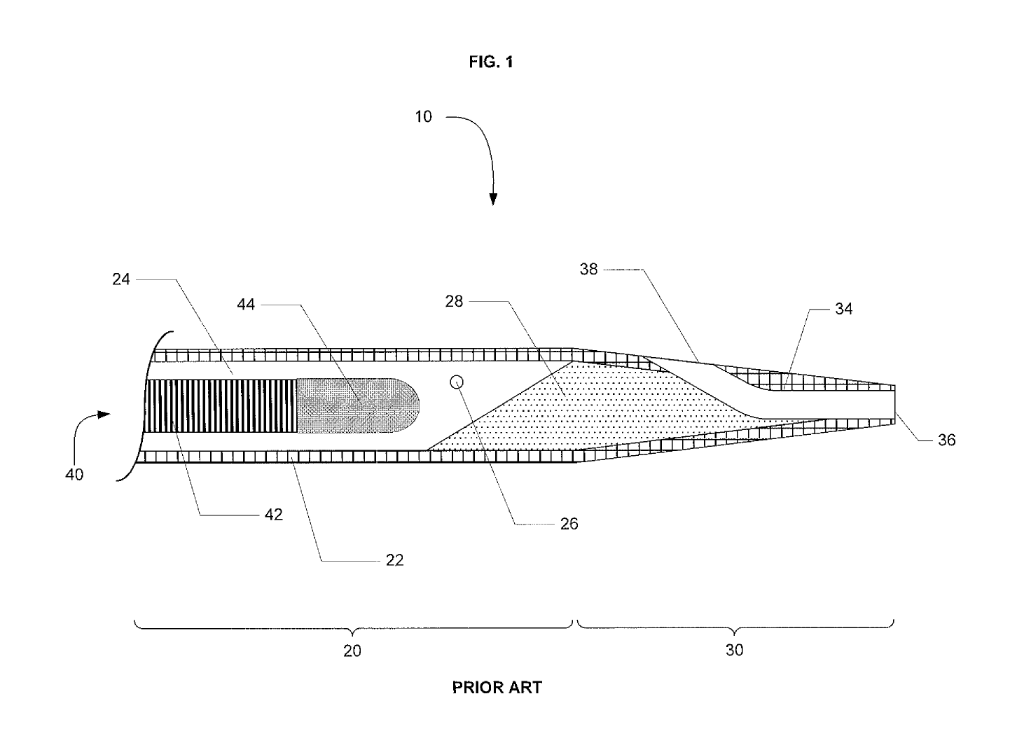

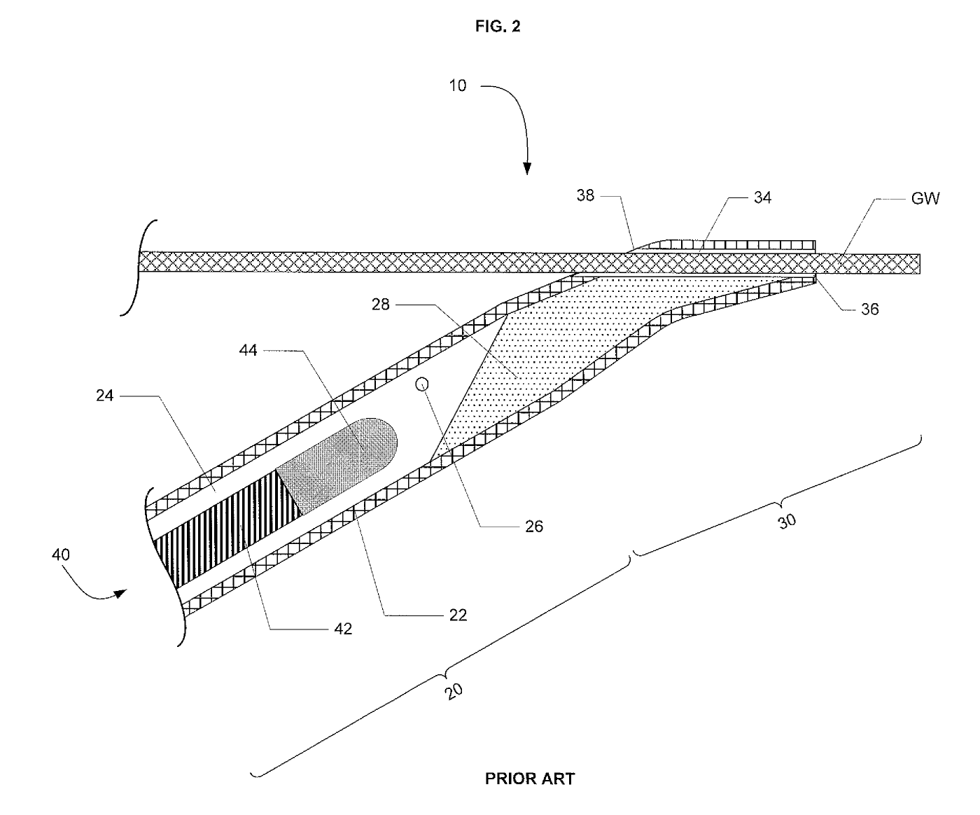

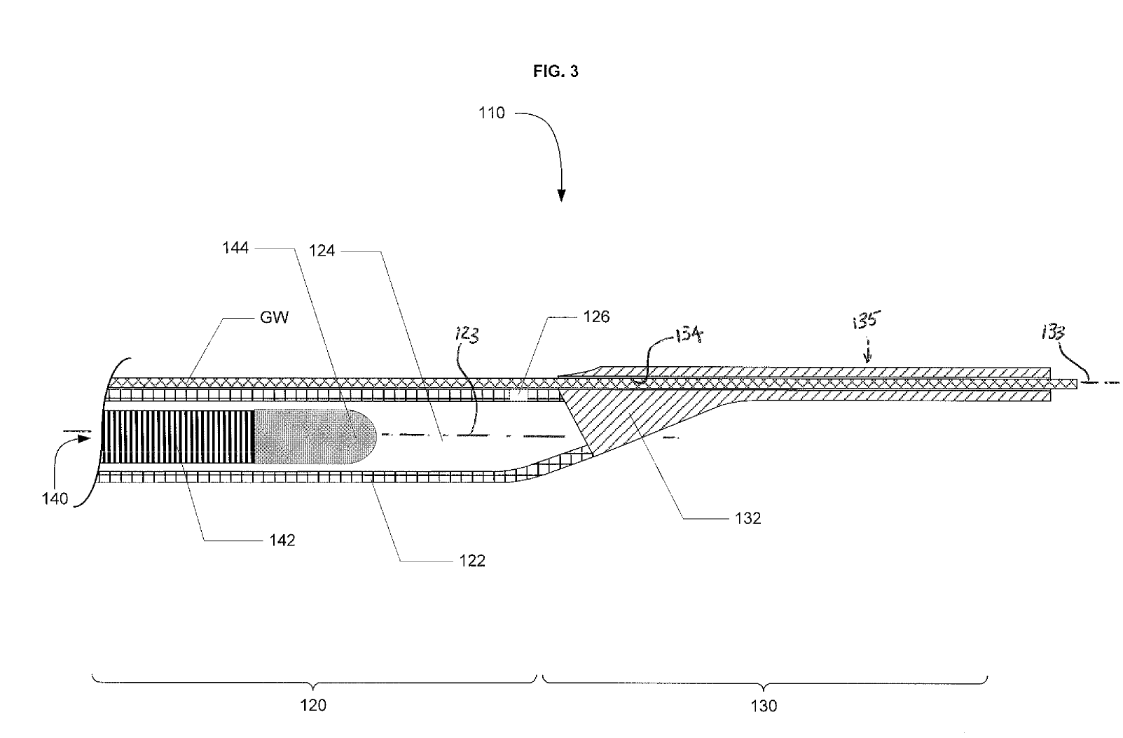

Referring now to FIGS. 1-3, an exemplary prior art catheter distal section 10 comprises a guide wire lumen 34 that is non-parallel to the distal shaft 20. When a guide wire GW is inserted through the guide wire lumen 34 of the distal tip 30, the distal tip and guide wire lumen bend to the shape of the stiffer guide wire. The bending sets up stresses in the distal section 30 and increases friction at the guide wire entry port 36 and exit port 38. The stresses are set up toward the outer side 37 of the entry port and inner side 39 of the exit port when the catheter is constrained by a small coronary artery wall 2. The increased friction makes the catheter more difficult to navigate to the target site. The bending of the distal section 30 further increases the profile of the distal section and guide wire, particularly the distal end of the distal shaft 20. The increased profile of the catheter and guide wire makes it more difficult to reach more distal sections of vessels having smaller lumen diameters The angled guide wire lumen is reinforced by additional material 28 and may be prone to prolapse (or kinking) when traversing tortuous paths. The reinforcement material also increases the distance between the distal end of the working lumen and the distal tip.

Among many advantages, the present invention provides for reduction of friction between the catheter distal tip and guide wire, reduction in profile of the catheter and guide wire, and increased kink resistance. The present invention further provides for improved performance of imaging catheters.

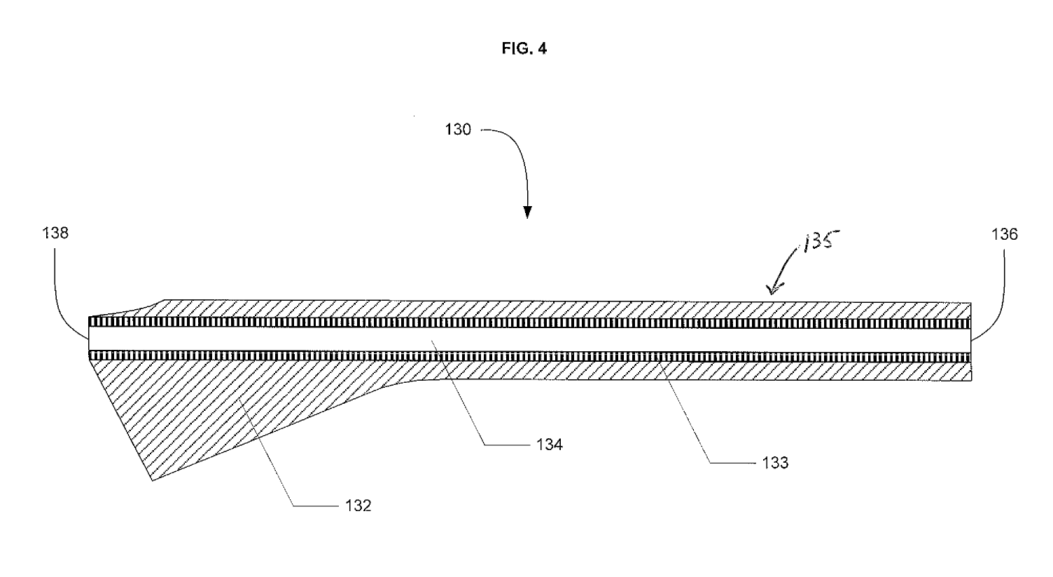

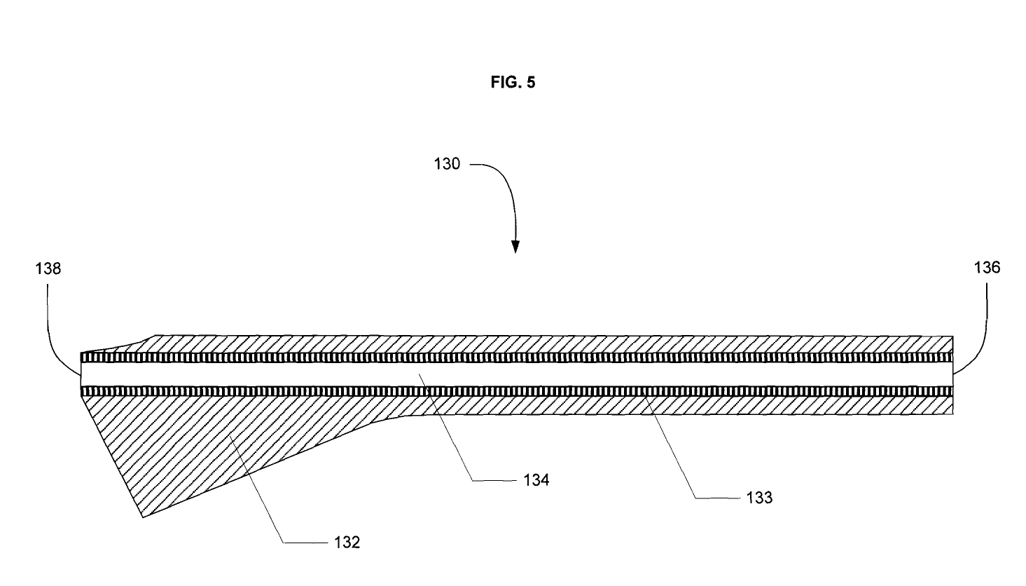

Referring now to FIGS. 4 and 5, a distal section and an enlarged view of a distal tip, respectively, of a monorail catheter according to one embodiment of the present invention are shown. The distal section 110 comprises a distal shaft 120 bonded to a distal tip section 130. The distal tip section 130 has a length of 15 mm or less and includes a distal tip 135. The longitudinal axis 133 of the distal tip 135 and its distal tip lumen 134 is substantially parallel to and offset from the longitudinal axis 123 of the distal shaft 120 and a distal shaft lumen 124 the distal shaft 120. The distal shaft lumen may have a working element 140 at or near its distal end. The following description of this exemplary embodiment will be directed to deployment in an imaging catheter where a distal shaft has a rotatable ultrasonic imaging core that is suitable for imaging of coronary arteries. More specifically the catheter may include an ultrasonic imaging core comprising a flexible drive cable 142 having a transducer housing and assembly 144 at its distal end. In an alternative embodiment, the working element may include an optical imaging core comprising a flexible drive cable bonded to an optical lens housing and assembly at its distal end. In other embodiments, the working element may be a mechanically rotatable combined ultrasound and optical imaging core.

The distal shaft 120 comprises an elongated tube 122 having at least one layer. The following description of the distal shaft will be directed at the case wherein the distal shaft is a straight nylon tube having a constant inner diameter in the range 0.024″ to 0.034″, generally 0.032″. The outer diameter must be sufficiently small to travel through a 6 F guide catheter. The distal shaft wall thickness is generally 0.005″.

The distal tip 130 comprises an elongated tube having at least one layer 132. Pebax has been found to be a suitable material for the distal tip. The following description of the distal tip will be directed at the case wherein the distal tip comprises Pebax having a low-friction liner 133. Etched PTFE has been found to be a suitable material for the low-friction liner. Other low-friction materials such as thin-walled HDPE and polyester could be suitable replacements for the etched PTFE lining. The distal tip 130 is bonded to the distal shaft 120 having the working lumen 124 substantially parallel and offset to the guide wire lumen 134.

The etched PTFE lining has an inner diameter suitable for 0.014″ guide wire, generally an inner diameter of 0.0175″. The wall thickness is generally 0.001″. The Pebax section has an inner diameter in the range of 0.018″ to 0.030″, generally 0.024″. The Pebax section generally has a wall thickness of 0.003″. A platinum/iridium radiopaque marker band (not shown) may be included in the distal tip.

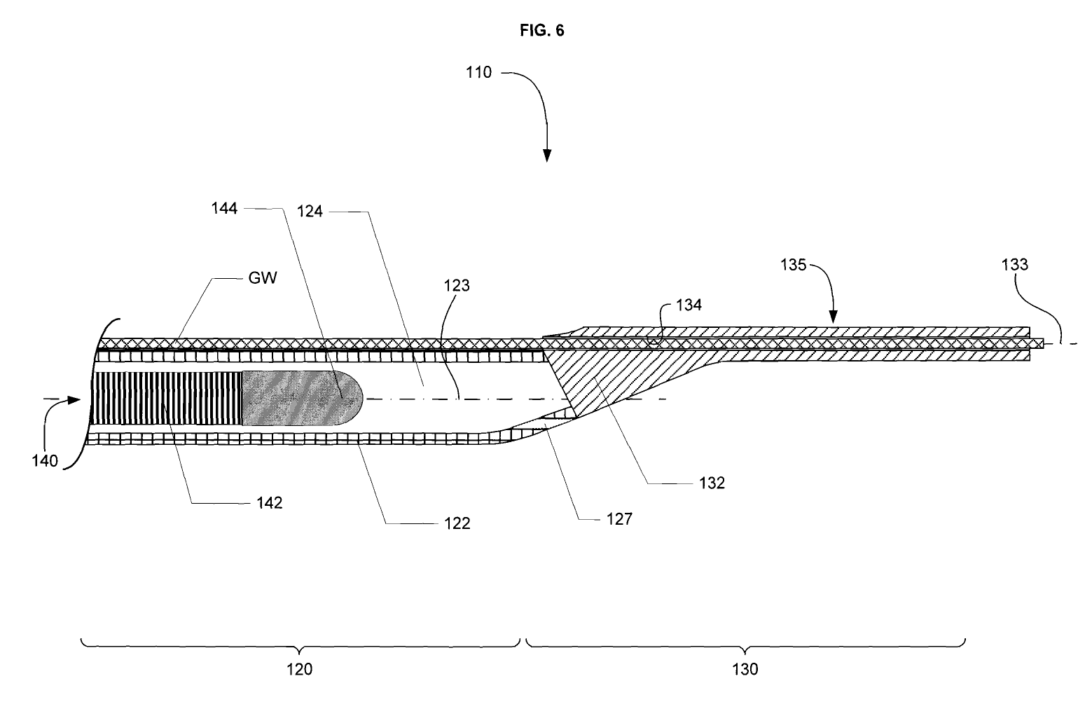

The guide wire exit port 138 is skived. At least one side flushing vent hole 126 for the distal shaft lumen is then punched at a range of 10 mm to 15 mm from the distal tip, generally 13 mm. The at least one side flushing vent hole 126 is formed adjacent to where the guide wire GW will be during use. With this arrangement, the guide wire GW will prevent a flushing stream from hitting a vessel wall. Other suitable positions for the vent hole comprise a forward flushing vent hole 127 at the distal end of the distal shaft as shown in FIG. 6. The distal section assembly can be further prepared for bonding to a suitable proximal shaft (not shown).

While particular embodiments of the present invention have been shown and described, modifications may be made, and it is therefore intended to cover in the appended claims, all such changes and modifications which fall within the true spirit and scope of the invention as defined by those claims.

What is claimed is:

1. In a catheter, a distal section comprising:
a distal tip section, the distal tip section having a distal tip and the distal tip having a longitudinal axis; and
a distal shaft, the distal shaft having a longitudinal axis,
wherein the distal tip longitudinal axis is substantially parallel to and offset from the distal shaft longitudinal axis.

2. The catheter of claim 1, wherein the distal shaft is formed from at least one layer of material.

3. The catheter of claim 1, wherein the distal shaft includes a lumen for containing a working element.

4. The catheter of claim 3 wherein the working element comprises a mechanically rotatable ultrasound imaging core.

5. The catheter of claim 3 wherein the working element comprises a mechanically rotatable optical imaging core.

6. The catheter of claim 3 wherein the working element comprises a mechanically rotatable combined ultrasound and optical imaging core.

7. The catheter of claim 1, wherein the distal tip section includes a guide wire lumen for receiving a guide wire.

8. The catheter of claim 7, wherein the longitudinal axis of the distal tip is also the longitudinal axis of the guide wire lumen.

9. The catheter of claim 7, wherein the distal shaft includes at least one flushing vent hole arranged to be adjacent the guide wire to enable the guide wire to prevent a flushing stream from the flushing vent hole from impinging a sidewall of a vessel in which the catheter is used.

10. The catheter of claim 7, wherein the distal shaft includes at least one forward flushing vent hole to prevent a flushing stream from the flushing vent hole from impinging a sidewall of a vessel in which the catheter is used.

11. The catheter of claim 1, wherein the distal tip section has a length of 15 mm or less.
